(19) United States
(12) Patent Application Publication
(10) Pub. No.: US 2005/0171080 A1
(43) Pub. Date: Aug. 4, 2005

(54) Polymorphs of ezetimibe and process for preparation thereof

(75) Inventors: Venkataraman Sundaram, Hyderabad (IN); Srinivasam Thirumalai Rajan, Hyderabad (IN); Vaddadi Pattabhi Ramayya, Hyderabad (IN); Sunkara Vishnu Vardhan, Ranga reddy District (IN); Bulusu Subrahmanyam, W. Godavari District (IN); Cheemalapati Venkata Annapurna Sasikala, Visakhapatnam Pin. (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(73) Assignee: Dr. Reddy's Laboratories, Inc., Bridgewater, NJ (US)

(21) Appl. No.: 11/022,570

(22) Filed: Dec. 23, 2004

(30) Foreign Application Priority Data
Dec. 23, 2003 (IN) 1049/CHE/2003

(51) Int. Cl. A61K 31/397; C07D 25/02

(52) U.S. Cl. 514/210.02; 540/200

(57) ABSTRACT

The present invention relates to novel crystalline forms and amorphous form of Ezetimibe and the processes for the preparation thereof.

The present application claims benefit of a filing date of an Indian Patent Application No. 1049/CHE/2003, filed Dec. 23, 2003, the contents of which are expressly incorporated herein by reference.

### BACKGROUND

The present invention relates to polymorphs of (3R, 4S)-1-(4-fluorophenyl)-3-[(3S)-3-(4-fluorophenyl)-3-hydroxypropyl]-4-(4-hydroxyphenyl)-2-azetidinone, which is generically known as Ezetimibe, and processes for the preparation thereof.

Ezetimibe is a cholesterol absorption inhibitor, for the treatment of hypercholesterolemia and may be used as combination therapy with Statin compounds such as atorvastatin, simvastatin, pravastatin, and lovastatin.

It is known that different polymorphic forms of a same drug may have substantial differences in certain pharmaceutically important properties. The amorphous form of a drug may exhibit different dissolution characteristics and in some case different bioavailability patterns compared to crystalline forms. See, e.g., Konne T., Chem. Pharm. Bull. 38, 2003 (1990). For some therapeutic indications one bioavailability pattern may be favored over another. For example, the amorphous form of cefuroxin axetil exhibit higher bioavailability than its crystalline form. Further, amorphous and crystalline forms of a drug may have different handling properties, dissolution rates, solubility, and stability. For these reasons, among others, access to a choice between the amorphous or crystalline forms of a drug is desirable for different applications. Therefore, there is a need for new solid forms of Ezetimibe and new methods of preparation.

### SUMMARY OF THE INVENTION

In accordance with one aspect, the present invention provides new crystalline Forms I and II of Ezetimibe. Preferably, the crystalline Form I of Ezetimibe has an X-ray diffraction pattern that contains peaks at about 13.8±0.1, 15.8±0.1, 24.5±0.1, and 26.3±0.1 and the crystalline Form II of Ezetimibe has an X-ray diffraction pattern containing peaks at about 7.9±0.1, 22.9±0.1, and 23.4±0.1. In accordance with another aspect, the present invention also provides an amorphous form of Ezetimibe.

In accordance with one aspect, the invention provides processes for preparing the new crystalline Forms I and II as well as the amorphous form. Preferably, the process for the Form I includes a) reacting 3-{2-[3-(fluorophenyl)-3-(trimethyl silyloxy)-propyl]-3-(4-fluoro phenyl amino)-3-(4-trimethyl silyloxy phenyl)-1-oxo-propyl}-4-(S)-phenyl oxazolidin-2-one with bistrimethyl silyl acetamide; b) quenching the reaction solution of step (a); c) adding sulfuric acid in an alcoholic solvent to the quenched reaction solution; d) isolating solid Ezetimibe; and e) drying the isolated solid Ezetimibe aerially to afford the crystalline Form I of Ezetimibe. The process for preparing the Form II includes providing pressure to the crystalline Form I.

In accordance with yet another aspect, the invention provides pharmaceutical compositions containing one or more pharmaceutically acceptable excipients and a prophylactically or therapeutically effective amount of individually or as mixtures in any proportions of the crystalline Forms I and II and the amorphous form of Ezetimibe. Also, the invention provides a method of treating or preventing a patient who has or potentially has a high cholesterol problem by administering the patient a prophylactically or therapeutically effective amount of individually or as mixtures in any proportions of the crystalline Forms I and II and amorphous form of Ezetimibe.

### BRIEF DESCRIPTION OF THE DRAWING

FIG. 1 shows an X-ray diffraction pattern of the crystalline Form I of Ezetimibe prepared by the inventors.

FIG. 2 shows an IR spectrum of the crystalline Form I of Ezetimibe prepared by the inventors.

FIG. 3 shows HPLC result of the crystalline Form I of Ezetimibe prepared by the inventors showing its purity.

FIG. 4 shows a thermogravimetry diagram of the crystalline Form I of Ezetimibe prepared by the inventors.

FIG. 5 shows a differential scanning calorimetry thermogram of the crystalline Form I of Ezetimibe prepared by the inventors.

FIG. 6 shows an X-ray diffraction pattern of Ezetimibe comprising the crystalline Form II prepared by the inventors.

FIG. 7 shows an IR spectrum of Ezetimibe comprising the crystalline Form II prepared by the inventors.

FIG. 8 shows HPLC result of Ezetimibe comprising the crystalline Form II prepared by the inventors.

FIG. 9 shows a thermogravimetry diagram of Ezetimibe comprising the crystalline Form II prepared by the inventors.

FIG. 10 shows a differential scanning calorimetry thermogram of Ezetimibe comprising the crystalline Form II prepared by the inventors.

FIG. 11 shows an X-ray diffraction pattern of the amorphous form of Ezetimibe prepared by the inventors.

FIG. 12 shows an IR spectrum of the amorphous form of Ezetimibe prepared by the inventors.

### DETAILED DESCRIPTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention the preferred methods and materials are described.

Unless stated to the contrary, any use of the words such as “including,” “containing,” “comprising,” “having” and the like, means “including without limitation” and shall not be construed to limit any general statement that it follows to the specific or similar items or matters immediately following it. Except where the context indicates to the contrary, all exemplary values are intended to be fictitious, unrelated to actual entities and are use for purposes of illustration only. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention as define by the appended claims.

The purpose of the present invention, the following terms are defined below.

The term, “pharmaceutically acceptable” means that which is useful in preparing a pharmaceutical composition that is generally non-toxic and not biologically undesirable and includes that which is acceptable for veterinary use and/or human pharmaceutical use.

The term, “patient” includes humans and animals.

“Therapeutically effective amount” means the amount of a compound that, when administered for treating or preventing a high cholesterol problem, is sufficient to effect such treatment or prevention in any recognizable degrees. The “therapeutically effective amount” may vary depending on the solid forms of Ezetimibe, and the patient's condition such as weight, age, severity of the cholesterol problem or risk, etc.

“Crystalline form I” and “crystalline form II” of Ezetimibe, mean novel crystalline forms of Ezetimibe that are invented by the present inventors. One of skill in the art would be able to distinguish these new crystalline forms from other known solid forms, if any, of Ezetimibe by reading, understanding, and comparing one or more the characterizations of the crystalline forms of the present invention described herein.

Ezetimibe is a compound of the formula:

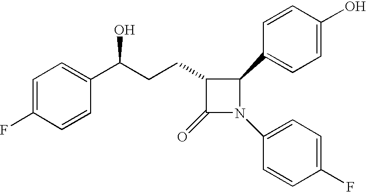

The process for the preparation of Ezetimibe is disclosed in U.S. Pat. No. 5,767,115.

According to one aspect, the invention provides two novel crystalline forms of Ezetimibe. The two crystalline forms obtained by the inventors are designated as Form Form I and II respectively.

The crystalline form I can be prepared by reacting 3-{2-[3-(fluorophenyl)-3-(trimethyl silyloxy)-propyl]-3-(4-fluoro phenyl amino)-3-(4-trimethyl silyloxy phenyl)-1-oxo-propyl}-4-(S)-phenyl oxazolidin-2-one with bistrimethyl silyl acetamide; quenching the reaction solution of the above; adding an acidic alcoholic solution to the quenched reaction solution; isolating solid Ezetimibe; and drying the isolated solid Ezetimibe aerially to afford the crystalline Form I of Ezetimibe. The quenching of the reaction solution may be done by any conventional methods such as adding acetic acid or small amount of water. The acidic alcoholic solution can be made by dissolving an acid such as sulfuric acid, HCl, etc. in an alcoholic solvent such as methanol, ethanol, etc. The crystallized Ezetimibe after acidifying the quenched reaction solution can be isolated by any conventional methods such as filtering. The aerial drying is done at room temperature for about 10 to 40 hours, preferably for about 20 to 24 hours.

The crystalline Form II can be prepared by providing pressure on the crystalline Form I. The pressure that may be employed to make the crystalline form II is about 4-7 ton/cm², preferably about 5-6 ton/cm². The pressure may be supplied for about 1-120 seconds, preferably about 30-60 seconds. Any equipments that are able to provide such pressure may be employed to make the crystalline Form II.

According to another aspect, the invention also provides amorphous form of Ezetimibe. The amorphous form of Ezetimibe may be prepared by dissolving Ezetimibe in an alcoholic solvent such as methanol, ethanol, etc. and removing the solvent under vacuum at the room temperature or less than about 70° C.

The crystalline Forms I and II as well as the amorphous form of Ezetimibe may be characterized by X-ray diffraction. X-ray diffraction patterns are unique for different crystalline forms. Each crystalline form may exhibit a diffraction pattern with a unique set of diffraction peaks that can be expressed in 2 theta angles, d-spacing values and relative peak intensities. 2 theta diffraction angles and corresponding d-spacing values account for positions of various peaks in the X-ray powder diffraction pattern. D-spacing values can be calculated with observed 2 theta angles and copper K (α1) wavelength using Bragg equation well known to those of skill in the art.

However, slight variation in observed 2 theta angles or d-spacing values are expected based on the specific diffractometer employed, preparation techniques and/or other experimental variations. More variation is expected for the relative peak intensities especially in low 2 theta angle region where is more sensitive to the crystalline sizes of the sample. Thus, identification of the exact crystal form of a compound should be based primary on observed 2 theta angles with lesser importance attributed to relative peak intensities.

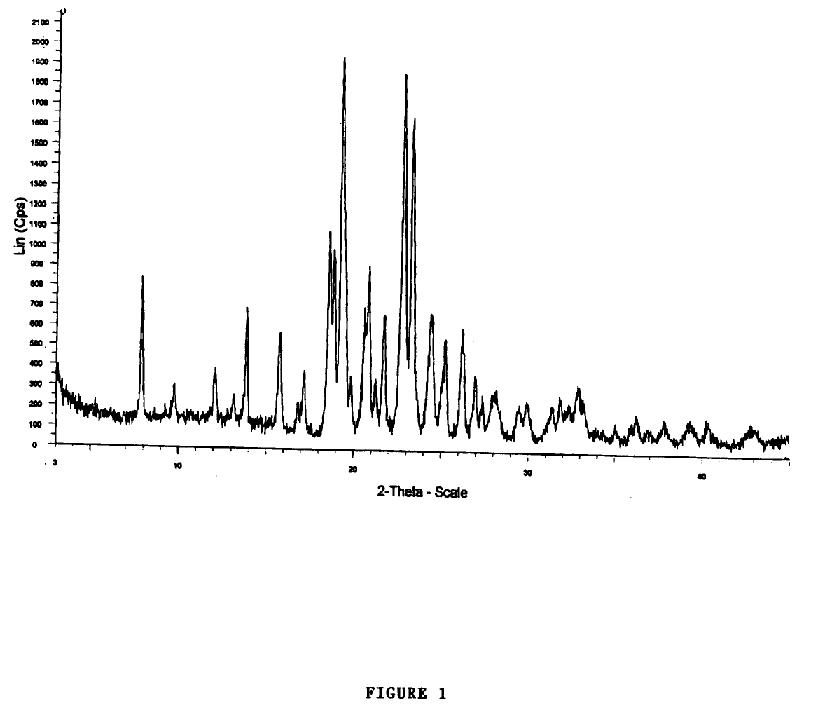

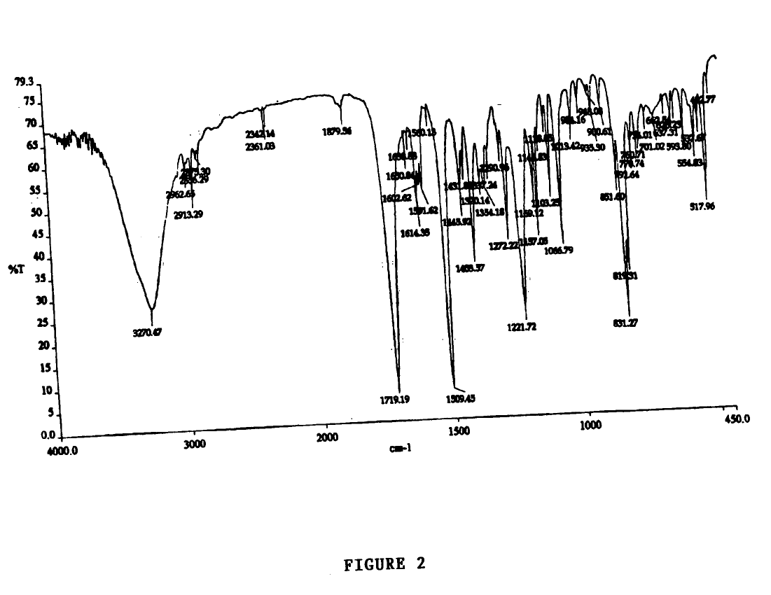

FIG. 1 shows an X-ray diffraction pattern of the crystalline Form I of Ezetimibe. FIG. 2 shows an X-ray diffraction pattern of Ezetimibe comprising the crystalline Form II. Both X-ray powder diffraction patterns were obtained on a Bruker Axs, D8 Advance Powder X-ray Diffractometer with CU K alpha-1 Radiation source.

Some margin of error is present in each of the 2 theta angle assignments reported herein. The assigned margin of error in the 2 theta angles for the crystalline Form I and Form II is ±0.1.

Although the entire peak pattern of an X-ray diffraction pattern may be used to identify a particular crystalline form, not all peaks are necessarily shown to prove presence of such crystalline form especially in a mixture of two or more difference crystalline forms. For example, proving presence of the crystalline Form I of Ezetimibe in a mixture of the crystalline Forms I and II does not require showing all the peaks of the X-ray diffraction pattern of the crystalline Form I in the X-ray diffraction pattern of the mixture. As long as the mixture is a mixture of only the Form I and Form II, and there is no other crystalline substance that attributes any peaks in the diffraction pattern, identifying one or two unique peaks that only exist in the pattern of the crystalline Form I would be sufficient to prove the presence of the crystalline Form I in the mixture.

Thus, understanding which peaks of a X-ray diffraction pattern of a crystalline form is distinctive over those of other crystalline form of the same compound would be particularly useful to determine presence of either form in the other form.

In case of the present invention, the X-ray diffraction patterns of the crystalline Forms I and II show that the peaks at about 7.9±0.1, 13.8±0.1, 15.8±0.1, 22.9±0.1, 23.4±0.1, 24.5±0.1, and 26.3±0.1 are particular useful for identification of the crystalline Form I over the Form II, and that the peaks at about 8.2±0.1, 13.6±0.1, 16.4±0.1, 20.2±0.1, and 29.7±0.1 of the crystalline Form I are especially distinctive over the peaks of the crystalline Form II.

A process for preparing an uncharacterized crystalline form of Ezetimibe is disclosed in U.S. Pat. No. 6,207,822. Since the crystalline form of the '822 patent was not characterized, the inventors of the present invention have repeated the '822 process to produce the uncharacterized crystalline form of Ezetimibe and characterize it. For convenience, the inventors have named it as crystalline Form X. The crystalline Form X of Ezetimibe has also been characterized by a Bruker Axs, D8 Advance Powder X-ray Diffractometer with CU K alpha-1 Radiation source, and the peaks form the obtained X-ray diffraction pattern is summarized along with the peaks of FIGS. 1 and 2 in Table 1.

| | | | Peaks taken from | | Peaks taken from | |
|---|---|---|---|---|---|---|
| | Form X | | FIG. 1 | | FIG. 2 | |
| S.No. | 2-Theta⁰ | Intensity % | 2-Theta⁰ | Intensity % | 2-Theta⁰ | Intensity % |
| 1. | 8.249 | 21.3 | 7.824 | 35.1 | 7.869 | 8 |
| 2. | 9.951 | 5.9 | 9.698 | 7.5 | 8.245 | 8.2 |
| 3. | 11.863 | 3 | 12.017 | 13.2 | 12.026 | 5.3 |
| 4. | 13.593 | 16.2 | 13.088 | 5.6 | 13.587 | 7.1 |
| 5. | 13.860 | 14.8 | 13.834 | 28 | 13.838 | 14.1 |
| 6. | 15.862 | 2 | 15.739 | 24.7 | 15.810 | 11.6 |
| 7. | 16.491 | 15.5 | 16.780 | 6.2 | 16.395 | 23 |
| 8. | 17.464 | 10.6 | 17.127 | 14.9 | 17.195 | 17.8 |
| 9. | 17.924 | 1.8 | 18.599 | 51.6 | 17.928 | 3.4 |
| 10. | 18.650 | 26.8 | 18.846 | 48.7 | 18.628 | 69.2 |
| 11. | 19.065 | 19.8 | 19.316 | 100 | 19.112 | 54.5 |
| 12. | 19.391 | 7.6 | 19.807 | 12.5 | 19.355 | 100 |
| 13. | 20.004 | 35.8 | 20.644 | 27.4 | 20.182 | 47.2 |
| 14. | 20.197 | 31.6 | 20.826 | 40.3 | 20.836 | 11.7 |
| 15. | 20.853 | 12.5 | 21.225 | 10.5 | 21.307 | 3.7 |
| 16. | 21.499 | 1.4 | 21.719 | 28.7 | 21.769 | 25.2 |
| 17. | 21.813 | 1.5 | 22.871 | 96.7 | 22.459 | 10.2 |
| 18. | 22.390 | 8.2 | 23.357 | 83.9 | 22677 | 13.3 |
| 19. | 22.982 | 8 | 24.450 | 31.4 | 22.894 | 20 |
| 20. | 23.593 | 100 | 25.254 | 24.2 | 23.567 | 37.5 |
| 21. | 23.931 | 18.2 | 26.263 | 27.6 | 23.894 | 18.9 |
| 22. | 25.593 | 42.6 | 26.990 | 14.2 | 25.312 | 21.6 |
| 23. | 26.211 | 3.5 | 27.387 | 6.6 | 25.580 | 11.7 |
| 24. | 27.346 | 1.2 | 28.144 | 9.5 | 26.250 | 5.7 |
| 25. | 28.006 | 6.7 | 29.487 | 7.1 | 27.018 | 8.3 |
| 26. | 29.722 | 21.3 | 29.966 | 9.3 | 27.311 | 5.2 |
| 27. | 30.191 | 6.9 | 31.404 | 6.3 | 28.031 | 10.4 |
| 28. | 30.690 | 4.2 | 31.884 | 8 | 29.713 | 18.4 |
| 29. | 31.277 | 1.7 | 32.406 | 5.9 | 30.140 | 5.5 |
| 30. | 31.815 | 1.5 | 32.930 | 10.8 | 31.403 | 3.9 |
| 31. | 33.874 | 4.1 | 33.257 | 6.7 | 32.883 | 4 |
| 32. | 34.321 | 2 | 36.277 | 5.6 | 36.290 | 3.6 |
| 33. | 35.131 | 3 | 37.870 | 4 | 37.140 | 2.9 |
| 34. | 36.066 | 2.7 | 39.368 | 4.8 | * | * |
| 35. | 37.105 | 3.9 | 40.400 | 4.3 | * | * |
| 36. | 38.438 | 2.1 | * | * | * | * |
| 37. | 44.514 | 2.1 | * | * | * | * |

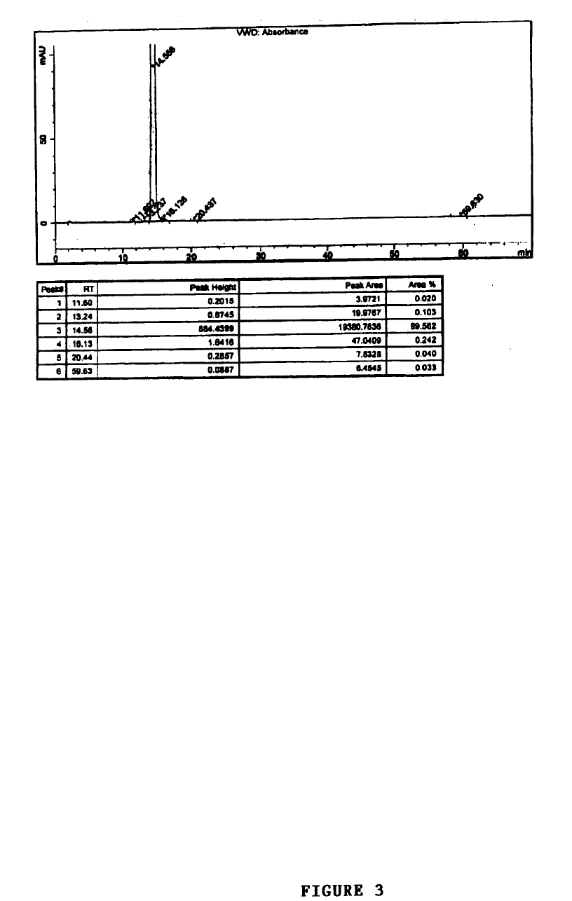

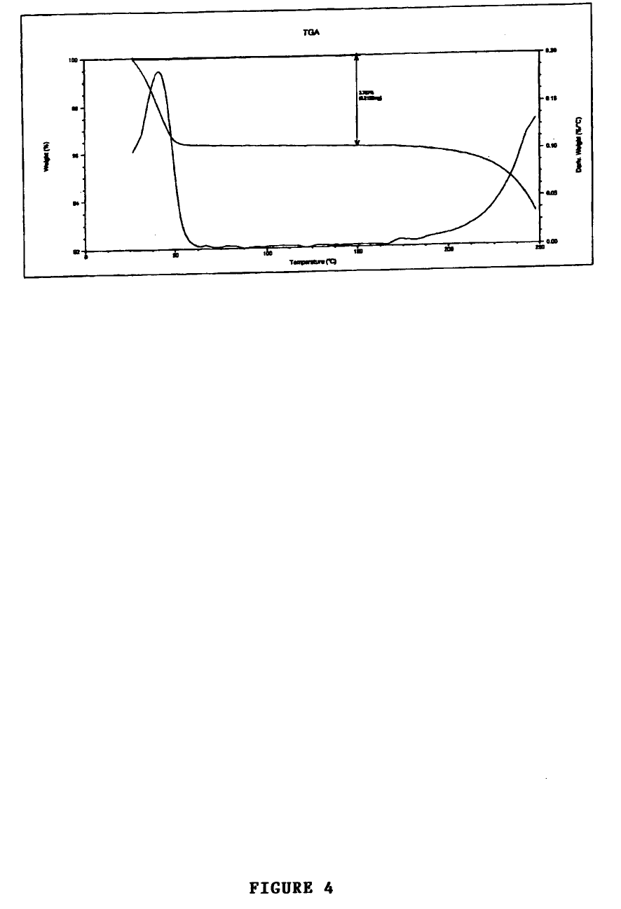

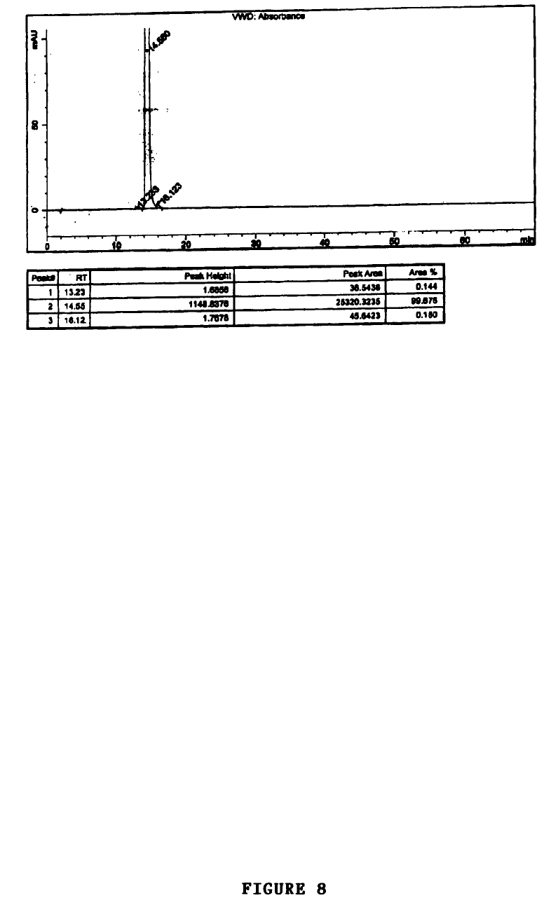

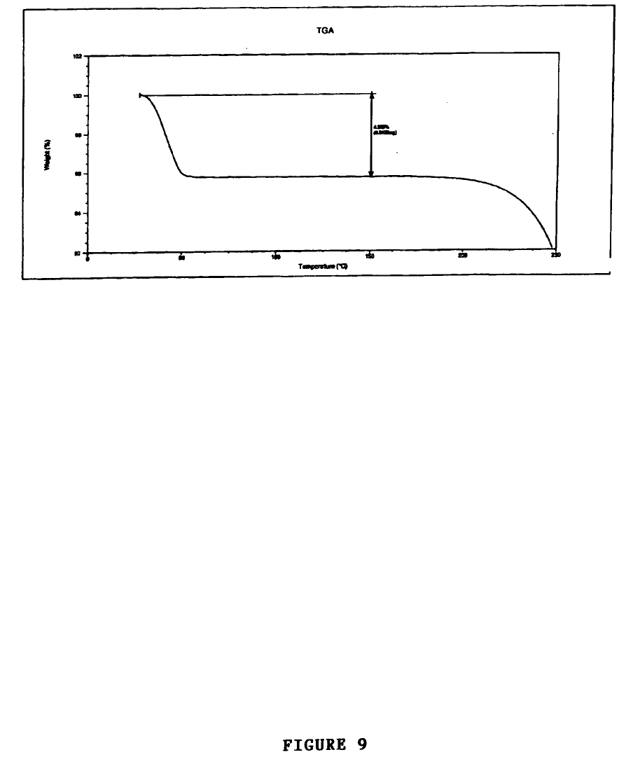

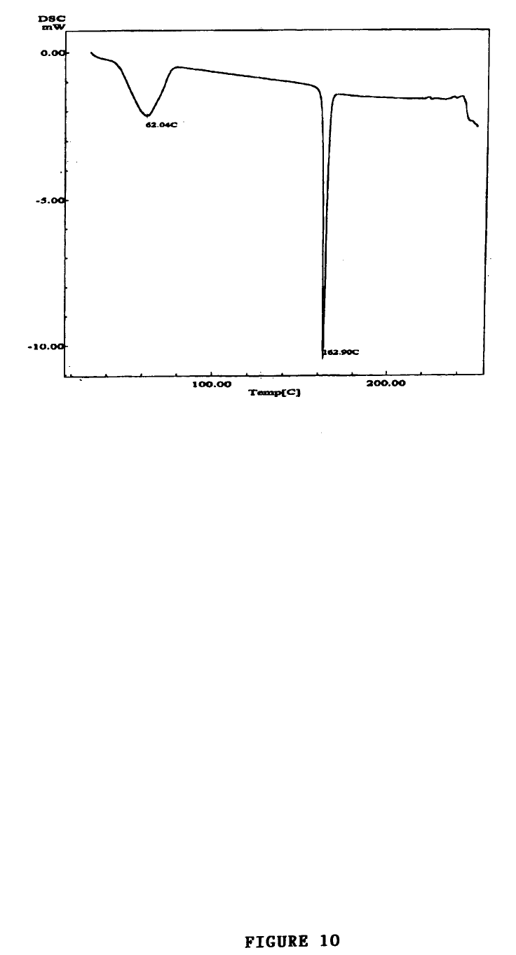

The crystalline Forms I and II of Ezetimibe are also characterized by IR, DSC, TGA, and HPLC, and their respective spectra are shown in FIGS. 2-5 and 6-10. The IR spectra were obtained by using Perkin-Elmer FT-IR instrument with KBr transmission method. The crystalline Form I shows a strong broad peak at about 3270 cm⁻¹, meanwhile the crystalline Form II shows consecutive peaks at 3438 and 3272 cm⁻¹. The DSC spectra show that the crystalline Form I has an endothermic peak at about 163° C. and the crystalline Form II at about 164° C.

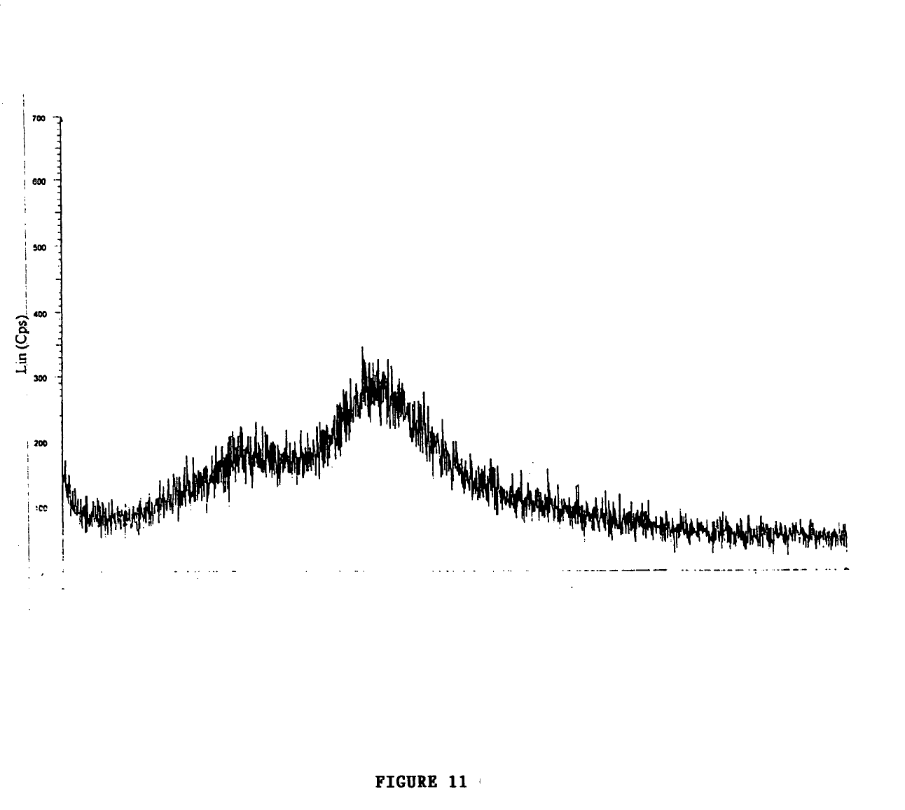

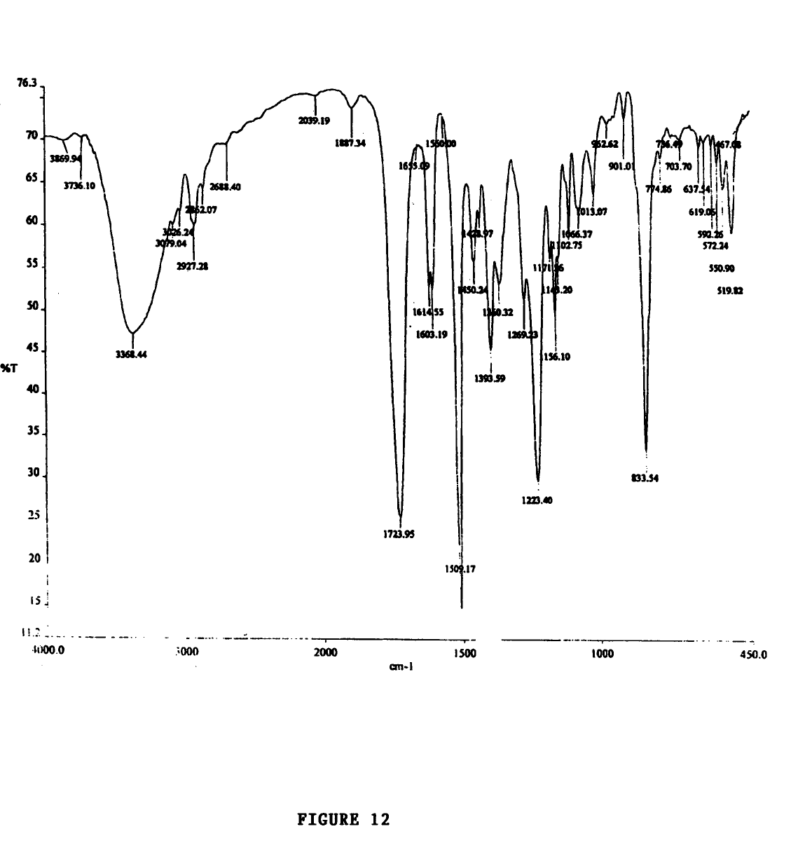

The present invention also provides the amorphous form of Ezetimibe. The process for preparing the amorphous form is also provided. The inventors concluded that amorphous, free-flowing form of Ezetimibe is useful in pharmaceutical processing. Advantages to using the amorphous form of Ezetimibe include enhance solubility. FIG. 11 shows the X-ray powder diffraction pattern of the amorphous form of Ezetimibe, which is also measured on a Bruker Axs, D8 Advance Powder X-ray Diffractometer with CU K alpha-1 Radiation source. The IR spectrum of the amorphous form is shown in FIG. 12.

In another aspect, the present invention provides a pharmaceutical composition containing, separately or as a mixture, the crystalline Forms I and II and the amorphous form of Ezetimibe and one or more pharmaceutically acceptable excipients. The pharmaceutical composition may be prepared by uniformly admixing the active ingredient with liquid or solid carriers and then shaping eh product into the desired form. The pharmaceutical compositions may be in the form of suspensions, solutions, elixirs, aerosols, or solid dosage forms. Because of their ease of administration, tablets and capsules represent the most advantageous oral dosage unit form, in which case solid pharmaceutical carriers are employed.

The invention is further illustrated by reference to the following examples describing in detail the preparation of the compound and the composition of the present invention, as well as their utility. However, the examples are not intended to limit any scope of the claim in anyway. It will be apparent to those skilled in the art that many modifications, both to materials, and methods, may be practiced without departing from the purpose and interest of this invention.

### Example 1

### Preparation of Crystalline Form I

Toluene (187 ml) was added to 3-{2-[3-(fluorophenyl)-3-(trimethylsilyloxy)-propyl]-3-(4-fluorophenylamino)-3-(4-trimethylsilyloxyphenyl)-1-oxo-propyl}-4-(S)-phenyl oxazolidin-2-one (25 g). Then added were bis-trimethylsillyl acetamide (15.1 ml) and Tetra butyl ammonium fluoride (1.3 g). The reaction mixture was maintained at 25-35° C. for 30-60 min and then was quenched with acetic acid. The solvent was distilled off under reduced pressure to give the crude compound, which was added to a mixture solution of methanol (38 ml) and 2N sulfuric acid solution (25 ml). After 30-60 min at 25-35° C., precipitated solids were filtered and washed with methanol (25 ml) and water (25 ml). Then, the filtered solids were aerially dried for 10-40 hours at 25-35° C. to give the crystalline Form I of Ezetimibe.

### Example 2

### Preparation of the Crystalline Form II

5-6 tonnes/cm² pressures were applied on Ezetimibe (5 gm), which was prepared from Example 1, by an IR press (Spartech) pellet maker for 30-60 seconds at room temperature to afford the crystalline Form II of Ezetimibe.

### Example 3

### Process for the Preparation of Amorphous Form of Ezetimibe

Toluene (187 ml) was added to 3-{2-[3-(fluorophenyl)-3-(trimethylsilyloxy)-propyl]-3-(4-fluorophenylamino)-3-(4-trimethylsilyloxyphenyl)-1-oxo-propyl}-4-(S)-phenyl oxazolidin-2-one (25 g). Then added bis-trimethylsillyl acetamide (15.1 ml) and Tetra butyl ammonium fluoride (1.3 g). Maintained the reaction mass at 25-35° C. for 30-60 min. The reaction mass quenched with acetic acid and distilled off the solvent under reduced pressure, to the crude compound added a mixture solution of methanol (38 ml) and 2N sulfuric acid solution (25 ml). Maintained the reaction mass for 30-60 min at 25-35° C. Filtered and washed the product with methanol (25 ml) and water (25 ml). Suck dried the compound for 15-30 min at 25-35° C. Taken the wet compound and dissolved in methanol (13 ml). Evaporated the solvent to dryness under vacuum. The compound was isolated by scratch to get the amorphous form of Ezetimibe (9.3 g).

### Example 4

### Preparation of Amorphous Form of Ezetimibe

Crystalline Form I of Ezetimibe (5 g) was dissolved in methanol (25 ml). The solvent was evaporated to dryness under vacuum at below 70° C. The compound was scratched to get amorphous form of Ezetimibe (4.5 g).

1. Crystalline Form I of Ezetimibe.

2. The crystalline form of claim 1, having an X-ray powder diffraction pattern with Cu K_{α1} radiation comprising peaks at about 13.8±0.1, 15.8±0.1, 24.5±0.1, and 26.3±0.1 degrees 2θ.

3. The crystalline form of claim 2, further comprising peaks at about 7.9±0.1, 22.9±0.1, and 23.4±0.1 degrees 2θ.

4. The crystalline form of claim 1, having an X-ray powder diffraction pattern substantially as shown in FIG. 1.

5. The crystalline form of claim 1, having an infrared absorption spectrum comprising a broad peak at about 3270 cm^{-1.}

6. The crystalline form of claim 1, having an infrared absorption spectrum substantially as shown in FIG. 2.

7. The crystalline form of claim 1, having an endothermic absorption peak at about 163° C. by differential scanning calorimetry.

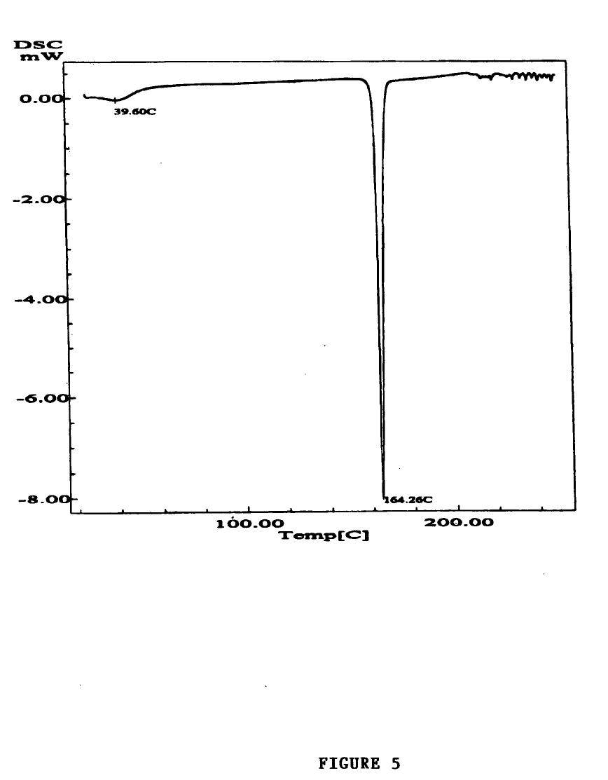

8. The crystalline form of claim 1, having a differential scanning calorimetry curve substantially as shown in FIG. 5.

9. A process for preparing crystalline Form I of ezetimibe, comprising:
a. reacting 3-{2-[3-(fluorophenyl)-3-(trimethyl silyloxy)-propyl]-3-(4-fluoro phenyl amino)-3-(4-trimethyl silyloxy phenyl)-1-oxo-propyl}-4-(S)-phenyl oxazolidin-2-one with bistrimethyl silyl acetamide;
b. quenching the reaction solution of step (a);
c. adding sulfuric acid in an alcoholic solvent to the quenched reaction solution; and
d. isolating solid Form I of Ezetimibe.

10. Crystalline Form I of Ezetimibe, prepared according to the process of claim 9.

11. A pharmaceutical composition comprising a prophylactically or therapeutically effective amount of the crystalline form of claim 1 and one or more pharmaceutically acceptable excipients.

12. A composition comprising crystalline Form II of Ezetimibe.

13. The crystalline form of claim 12, wherein said crystalline Form II has an X-ray diffraction pattern with Cu K_{α1} radiation comprising peaks at about 8.2±0.1, 16.4±0.1, 20.2±0.1, and 29.7±0.1 degrees 2θ.

14. The crystalline form of claim 13, wherein said peaks further comprise 13.6±0.1 degrees 2θ.

15. The crystalline form of claim 12, wherein said crystalline Form II has an infrared absorption spectrum comprising consecutive peaks at about 3438 and about 3272 cm^{-1.}

16. The crystalline form of claim 12, wherein said crystalline Form II has a differential scanning calorimetry curve having an endothermic peak at about 164° C.

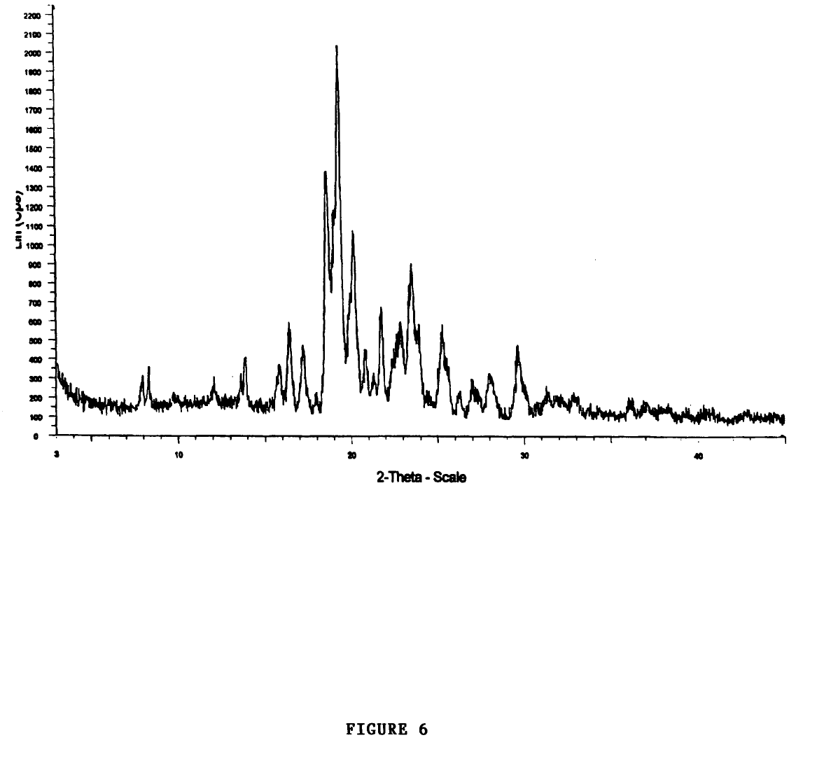

17. The crystalline form of claim 12, wherein said Ezetimibe has an X-ray diffraction pattern substantially as shown in FIG. 6.

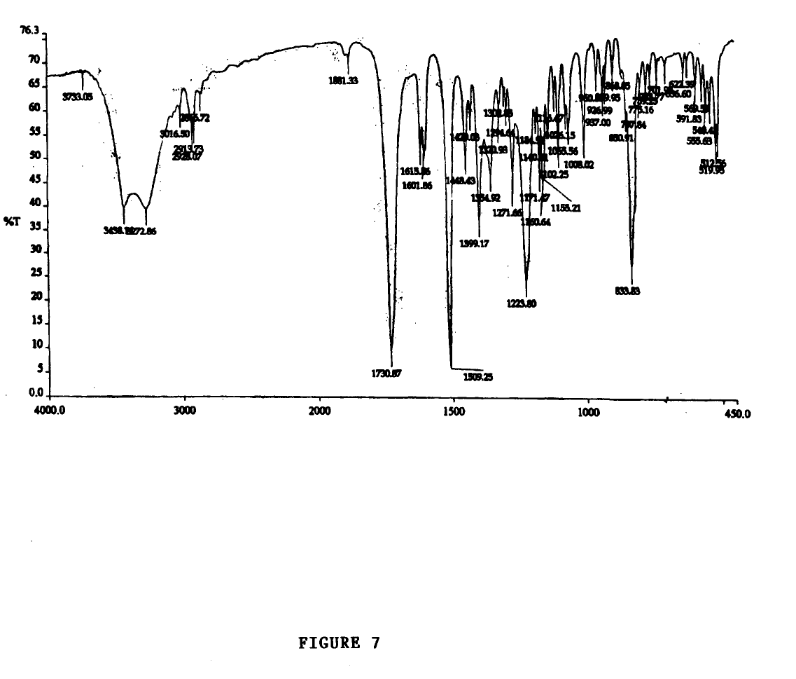

18. The crystalline form of claim 12, wherein said Ezetimibe has an infrared absorption spectrum substantially as shown in FIG. 7.

19. A process for preparing crystalline Form II of Ezetimibe comprising applying pressure to crystalline Form I of Ezetimibe.

20. The process of claim 19, wherein said pressure is between about 4-7 tonnes/cm².

21. The process of claim 19, wherein said pressure is between about 5-6 tonnes/cm².

22. The process of claim 19, wherein said pressure is provided for about 1-120 seconds.

23. The process of claim 19, wherein said pressure is provided for about 30-60 seconds.

24. Ezetimibe, which is prepared according to the process of claim 19.

25. A pharmaceutical composition comprising a prophylactically or therapeutically effective amount of the crystalline form of claim 12 and one or more pharmaceutically acceptable excipients.

26. A composition comprising crystalline Form I and crystalline Form II of Ezetimibe.

28. The amorphous form of claim 27, having an X-ray diffraction pattern substantially as shown in FIG. 11.

29. The amorphous form of claim 27, having an infrared absorption spectrum substantially as shown in FIG. 12.

30. A pharmaceutical composition comprising a prophylactically or therapeutically effective amount of the amorphous form of claim 27 and one or more pharmaceutically acceptable excipients.

31-33. (canceled)
